(19) United States
(12) Patent Application Publication
(10) Pub. No.: US 2019/0099082 A1
(43) Pub. Date: Apr. 4, 2019

(54) FIBER ASSEMBLY FOR RESPIRATORY GAS DETECTION

(71) Applicant: KONINKLIJKE PHILIPS N.V., EINDHOVEN (NL)

(72) Inventors: PETRUS THEODORUS JUTTE, WEERT (NL); ADRIANUS WILHELMUS DIONISIUS MARIA VAN DEN BIJGAART, HELVOIRT (NL); NICOLAAS LAMBERT, WAALRE (NL); HANS WILLEM VAN KESTEREN GIRO, EINDHOVEN (NL); ALEXANDER MARC VAN DER LEE, VENLO (NL)

(21) Appl. No.: 16/087,672

(22) PCT Filed: Mar. 23, 2017
(86) PCT No.: PCT/EP2017/056953

§ 371 Date: Sep. 24, 2018

Related U.S. Application Data
(60) Provisional application No. 62/312,154, filed on Mar. 23, 2016.

(51) Int. Cl. A61B 5/00; G01N 33/497; A61B 5/083; G01N 21/3504

(52) U.S. Cl. CPC A61B 5/0075; G01N 33/497; G01N 2021/0314; G01N 21/3504; G01N 2201/08; A61B 5/083

(57) ABSTRACT

A fiber assembly (60) for capnography or oxygraphy employing an housing (61), a collimator (64), a retroreflector (67) and a single mode optical fiber (63). Housing (61) including a respiratory gas detection chamber (62). Collimator (64) is rigidly disposed within or detachably attached to housing (61), and retroreflector (67) is rigidly disposed within or detachably attached to housing (61). Collimator (64) and retroreflector (67) are optically aligned within housing (61) across respiratory gas detection chamber (62). Optical fiber (63) is optically aligned with collimator (64) within or external to the housing (61). In operation, optical fiber (63) emits a gas sensing light beam through collimator (64) across respiratory gas detection chamber (62) to retroreflector (67), and optical fiber (63) receives a gas detection light beam reflected from retroreflector (67) across respiratory gas detection chamber (62) through collimator (64) to optical fiber (63). The gas detection light beam is indicative of the degree of carbon dioxide or oxygen within any gas flowing through respiratory gas detection chamber (62).

### FIELD OF THE INVENTION

The following relates generally to fiber assembly for respiratory gas detection, and more particularly to tolerance friendly single mode fiber assembly for capnography or oxygraphy and related methods of manufacture and use.

### BACKGROUND OF THE INVENTION

One type of respiratory gas detection is capnography, which is the monitoring of the concentration or partial pressure of carbon dioxide (CO₂) in respiratory gases. A known respiratory gas detection device is the Respironics® LoFlo® Sidestream CO₂ sensor available from Koninklijke Philips N. V., Eindhoven, the Netherlands, which uses a non-dispersive infrared (NDIR) single beam optical measurement technique to measure CO₂ in respiratory gas samples via a nasal cannula or other patient accessory. The LoFlo® CO₂ sensor includes a pump for drawing respiratory gas into a sample cell. Another type of respiratory gas detection is oxygraphy, which is the monitoring of the concentration or partial pressure of oxygen (O₂) in respiratory gases. Oxygraphy can be combined with Capnography for monitoring the metabolism of patients.

The present disclosure provides an alternative to the LoFlo® Sidestream CO₂ sensor. In particular, disclosed and described herein is a class of assemblies based on a light path with one or more optical fibers on one side of a respiratory gas detection chamber and an optical reflector on the other side of the respiratory gas detection chamber that have clear advantages for capnography and other gas detection applications.

One such advantage is that the use of optical fibers for transport of the optical radiation of the source and the detection light eliminates the need for a pump. In the LoFlo® side stream respiratory gas detection device from Philips Respironics, the pump is responsible for a large part of the cost of the unit and it consumes a significant amount of power inhibiting a low power mobile device. The use of optical fiber(s) also provides for having less cables around a patient's bed, no congestion problems in the sampling tube and no signal delay and distortion of the capnogram due to the gas transport.

In a mainstream configuration, optical fiber(s) can be used as well with the advantage that the bulky and heavy part of the CO₂ measurement unit can be placed away from the airway adapter, allowing for a light weight, comfortable sensor.

Fiber assemblies suitable for capnography or oxygraphy should be rigid, robust and low cost.

To date, there is no known respiratory gas detection device incorporating a fiber that exists which is able to properly function and be suitable for commercial use due to major problems and issues which heretofore have not been able to be overcome.

One of the major problems of a respiratory gas detection device incorporating a fiber is the rather high cost price of the optical assembly which is needed for measuring the CO₂ or O₂ rate.

In addition, test results have shown that the detection signal contains a significant disturbance due to mode interference when multi-mode fibers are applied. Single-mode fibers gave a much better result on the issue. However, the application of single-mode fibers result in very tight tolerances of the optical assembly being required, significantly increasing the costs to a level generally considered to be not practical for commercial use.

In tunable diode laser absorption spectroscopy (TDLAS), generally, a parallel light beam is used that enters and exits the gas cell through windows. Multipass cells are also known where the beam is reflected multiple times to enhance the absorption in the gas cell. The light path between source and windows is usually an open light path, but fiber-optic light paths are also known.

For example, FIG. 1A illustrates a fiber assembly 20 having a respiratory gas detection chamber 21 defining an exemplary light path for measuring gas concentrations in combination with an optical fiber 22 and an optical fiber 27. In operation, optical fiber 22 is illuminated by a light source (not shown). The light source is preferably a laser for proper light coupling efficiency and a wavelength suitable for either CO₂ or O₂ detection. The light beam from optical fiber 22 is collimated into a parallel beam by lens 23. Any CO₂ (O₂) in the air flowing through respiratory gas detection chamber 21 between windows 24 and 25 absorbs part of the light. The non-absorbed part of the beam is focused into optical fiber 27 by lens 26. Optical fiber 27 is connected to a detector (not shown). FIG. 1B illustrates exemplary spacing between components on a millimeter scale.

Experiments have shown that the detection signal contains a significant disturbance due to mode interference when multi-mode fibers are applied. Single-mode fibers gave a much better result on the issue. However, the application of single-mode fibers result in very tight tolerances of the optical assembly. Such tolerances were found to be in the order of magnitude of 1 μm and/or 0.1 mrad.

### SUMMARY OF THE INVENTION

To overcome the above mentioned problems and issues, discloses and described herein is an optical assembly where one or more single mode optical fibers is(are) coupled in from one side of a respiratory gas detection chamber.

One embodiment of the inventions of the present disclosure is a fiber assembly for respiratory gas detection employing a housing, a collimator, a retroreflector and a single mode optical fiber. The housing including a respiratory gas detection chamber. The collimator is either rigidly disposed within or detachably attached to the housing, and the retroreflector is also either rigidly disposed within or detachably attached to the housing. The collimator and the retroreflector are optically aligned within the housing across the respiratory gas detection chamber. The single mode optical fiber is optically aligned with the collimator within or external to the housing for an emission of a gas sensing light beam by the single optical fiber through the collimator across the respiratory gas detection chamber to the retroreflector, and for a reception by the single mode optical fiber of a gas detection light beam reflected from the retroreflector across the respiratory gas detection chamber through the collimator to the single mode optical fiber. The gas detection light beam is indicative of the degree of carbon dioxide or oxygen within any gas flowing through the respiratory gas detection chamber as known in the art of the present disclosure.

A second embodiment of the inventions of the present disclosure is a respiratory gas detection device employing the fiber assembly and an optical control assembly optically coupled to the single mode optical fiber. The optical control assembly includes a laser for generating the gas sensing light beam, a light detector for detecting the gas detection light beam, and an optical fiber circulator structurally configured to direct the gas sensing light beam from the laser to the single mode optical fiber and to direct the carbon dioxide sampled light beam from the single mode optical fiber to the light detector.

For purposes of describing and claims the inventions of the present disclosure, the terms “single mode optical fiber”, “collimator”, “retroreflector”, “mirror”, “lens”, “laser”, “light detector” and “circulator” are to be interpreted as known in the art of the present disclosure and exemplary described herein.

More particularly, a single mode optical fiber broadly encompasses all optical fibers, as known in the art of the present disclosure and hereinafter conceived, in which only the lowest order bound mode can propagate at the wavelength of interest.

A collimator broadly encompasses any device, as known in the art of the present disclosure and hereinafter conceived, for making collimated (parallel) light. A non-limiting example of a collimator is a GRIN lens as known in the art of the present disclosure.

A retroreflector broadly encompasses any device, as known in the art of the present disclosure and hereinafter conceived, having a surface for non-scattering/insignificant scattering reflection of light back to a source of the light. Non-limiting examples of a retroreflector include a corner reflector, a prism reflector, a cone reflector and a cat's eye.

Also for purposes of describing and claims the inventions of the present disclosure, the term “gas sensing light beam” broadly encompasses a light beam emitted from an optical fiber for purposes of passing the light beam through a gas containing an unknown degree of carbon dioxide or oxygen, and the term “gas detection light beam” broadly encompasses a carbon dioxide or oxygen detection sampling light beam received by the optical fiber after passing through the gas containing the unknown degree of carbon dioxide or oxygen.

The foregoing embodiments and other embodiments of the inventions of the present disclosure as well as various features and advantages of the inventions of the present disclosure will become further apparent from the following detailed description of various embodiments of the inventions of the present disclosure read in conjunction with the accompanying drawings. The detailed description and drawings are merely illustrative of the inventions of the present disclosure rather than limiting, the scope of the inventions of the present disclosure being defined by the appended claims and equivalents thereof.

### BRIEF DESCRIPTION OF THE DRAWINGS

FIGS. 1A and 1B illustrate an exemplary embodiment of a fiber assembly having a straight light path from a light emitting optical fiber to a light receiving optical fiber as known in the art of the present disclosure.

FIGS. 2A-2F illustrate exemplary embodiments of a fiber assembly having a folded light path from a light emitting optical fiber to a light receiving optical fiber in accordance with the inventive principles of the present disclosure.

FIGS. 3A-3S illustrate exemplary embodiments of a fiber assembly having a reflected light path between an optical fiber and a retroreflector in accordance with the inventive principles of the present disclosure.

FIGS. 4A-4F illustrates exemplary embodiments of a fiber assembly in accordance with the inventive principles of the present disclosure.

FIG. 5 illustrates an exemplary embodiment of a respiratory gas detection device in accordance with the inventive principles of the present disclosure.

FIG. 6 illustrates an exemplary mounting of a fiber assembly onto a patient in accordance with the inventive principles of the present disclosure.

### DETAILED DESCRIPTION OF THE PREFERRED EMBODIMENTS

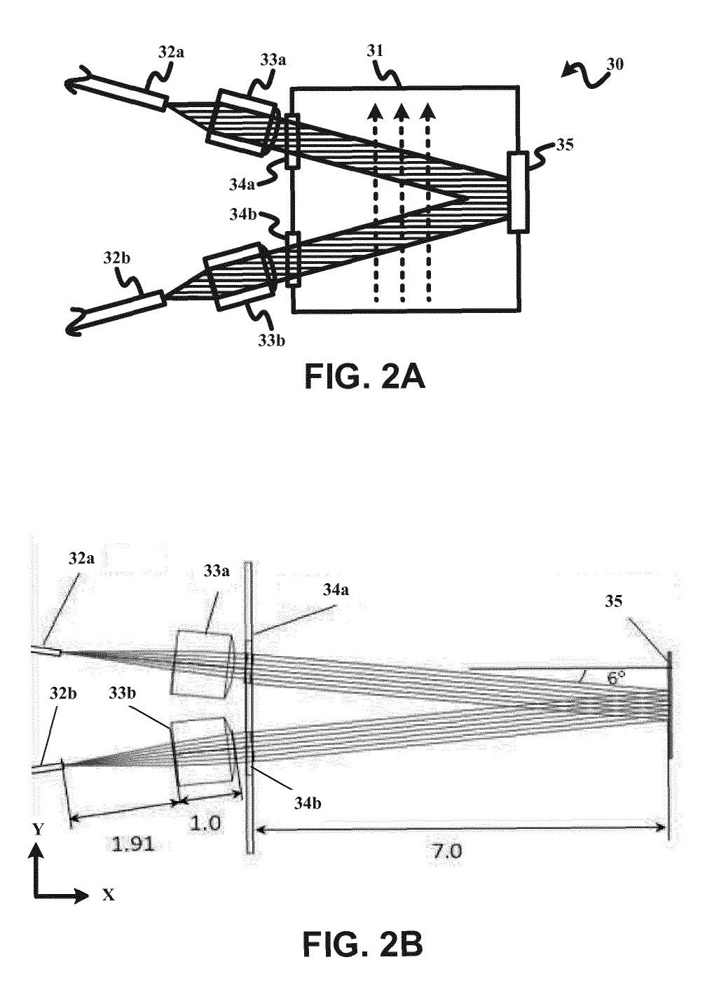

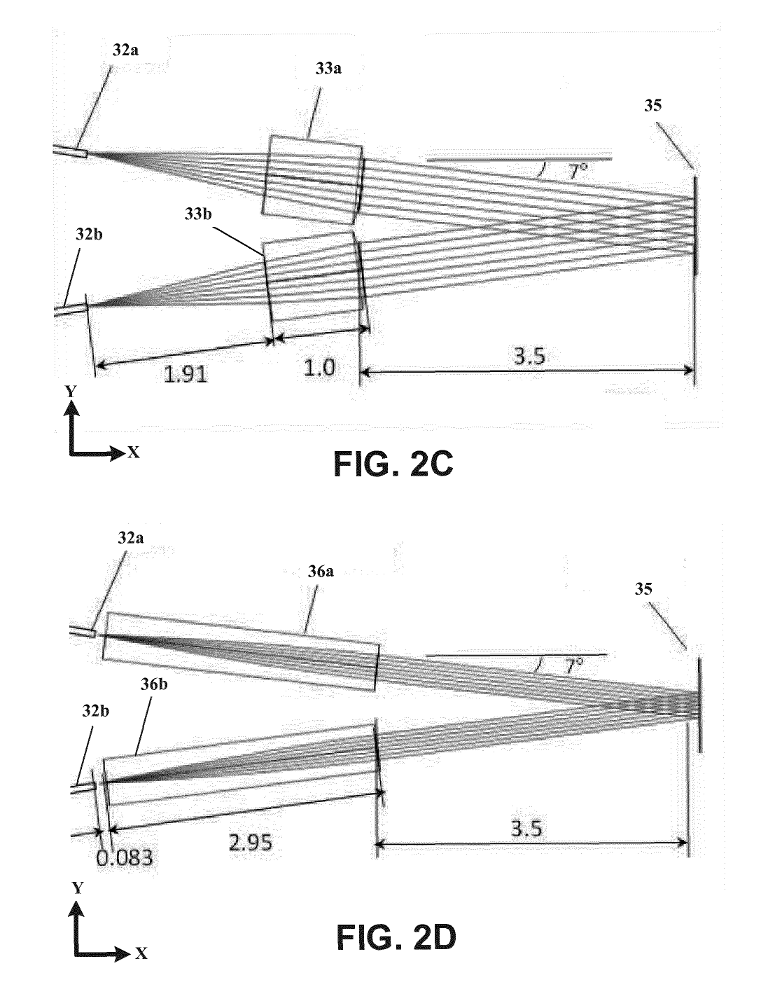

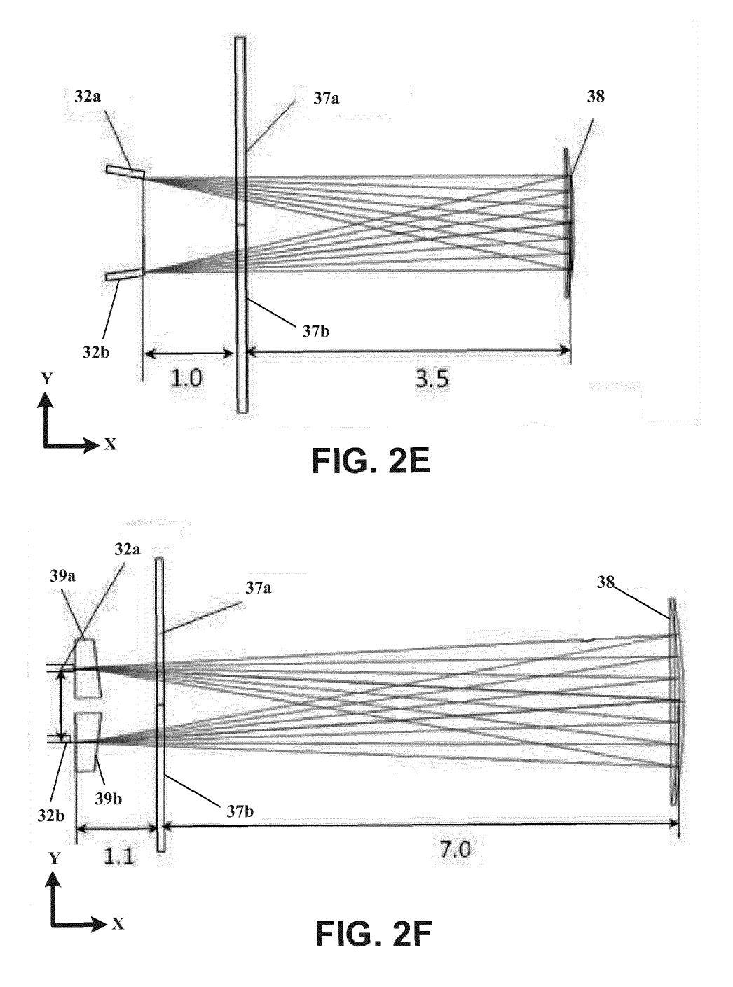

To facilitate an understanding of the inventions of the present disclosure, the following description of FIGS. 2A-2F teaches basic inventive principles of a fiber assembly having a folded light path from an emitting optical fiber to a receiving optical fiber in accordance with the inventive principles of the present disclosure. From this description of FIG. 2A-2F, those having ordinary skill in the art will appreciate how to apply the inventive principles of the present disclosure to practice numerous and various embodiments of a fiber assembly having a folded light path from a emitting optical fiber to a receiving optical fiber in accordance with the inventive principles of the present disclosure.

Referring to FIG. 2A, a fiber assembly 30 employs a light emitting single mode optical fiber 32a and a light receiving single mode optical fiber 32b. In operation, a gas containing a degree of carbon dioxide flows through respiratory gas detection chamber 31 as exemplary symbolized by the upwardly pointing dashed arrows. Optical fiber 32a emits a gas sensing light beam sequentially through an (a)spherical lens 33a and a window 34a across respiratory gas detection chamber 31 to a flat mirror 35 whereby a gas detection light beam is reflected back across respiratory gas detection chamber 31 sequentially through a window 34b and an (a)spherical lens 33b to optical fiber 32b. The gas detection light beam is indicative of the degree of carbon dioxide or oxygen within any gas flowing through the respiratory gas detection chamber known in the art of the present disclosure.

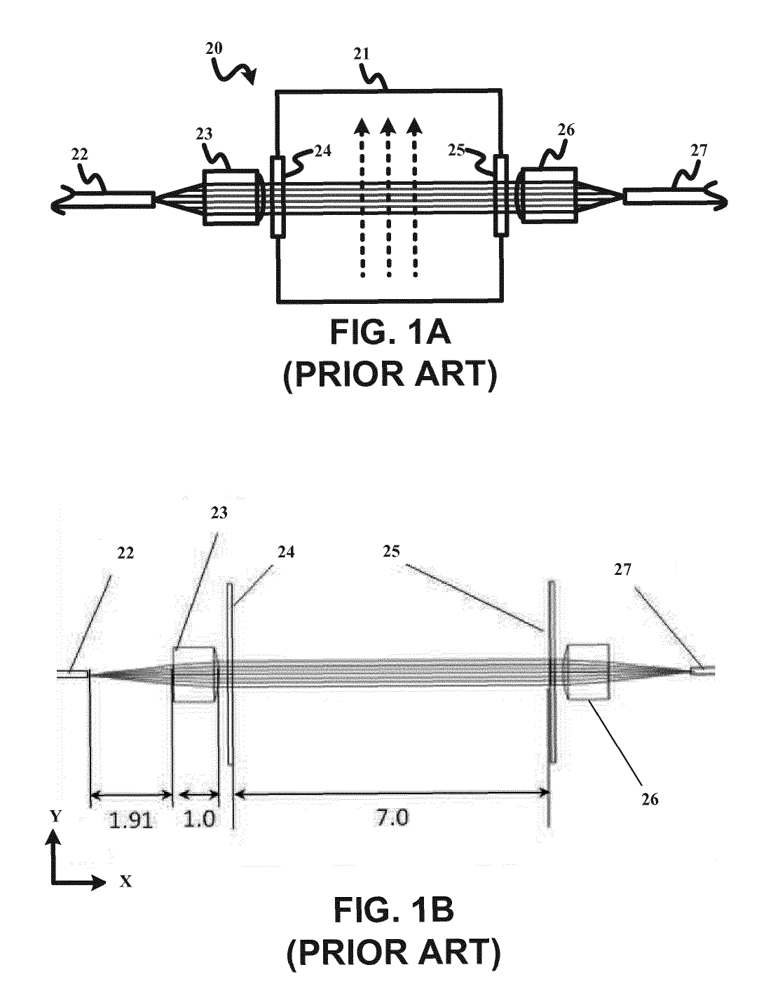

FIGS. 2B and 2C illustrate exemplary dimensional spacing for fiber assembly 30 as shown in 2A. In particular, FIG. 2C illustrates the dimensions may be reduced without sacrificing the measurement length (windows 34 and 36 not shown) while keeping the absorption path length the same as FIG. 1B.

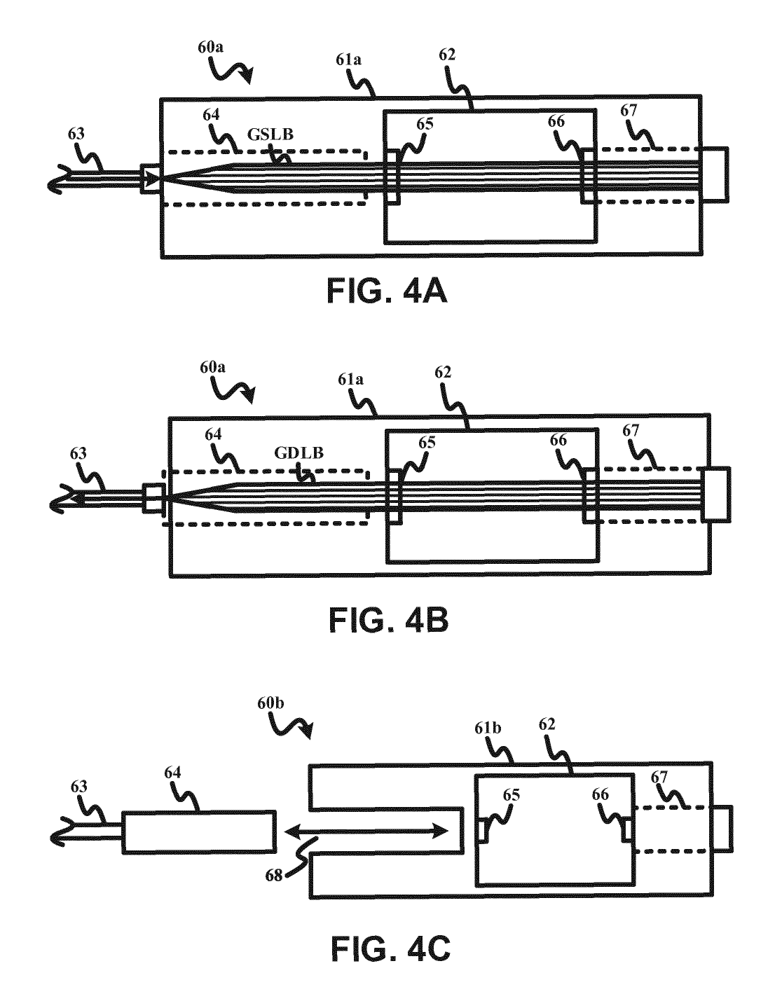

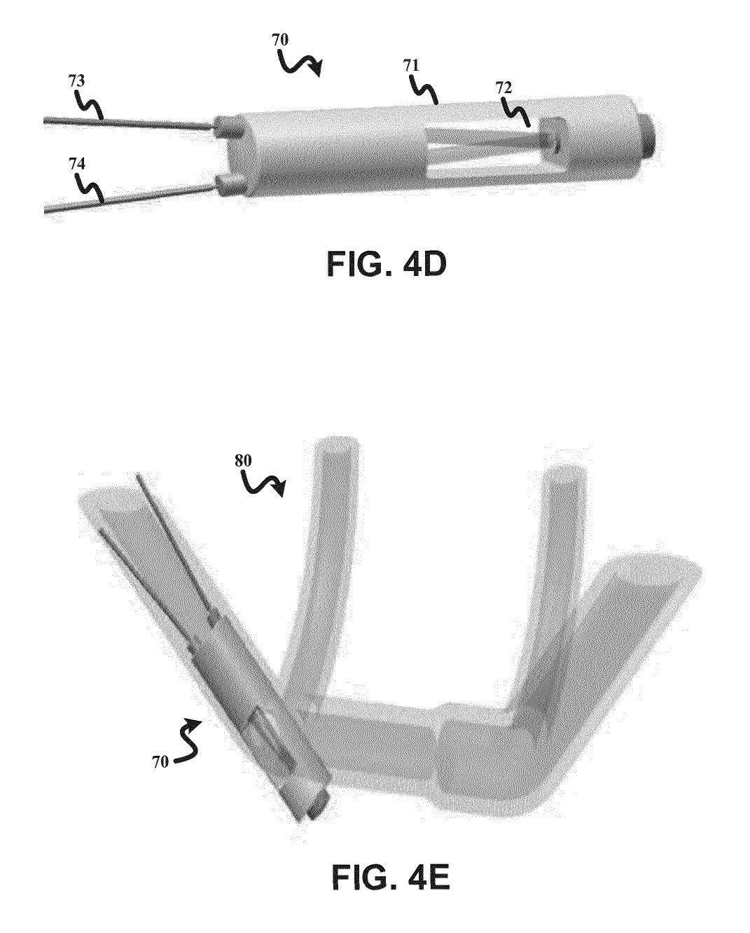

Optical assemblies in accordance with the present disclosure, such as disclosed in FIGS. 2A-2C, for example, enable a mechanical stable construction because the compactness of the housing results in a stiff system as will be further described herein in connection with FIGS. 4A-4E.

This advantage of the present disclosure is in contrast with a ‘cross over’ system such as shown in FIGS. 1A and 1B, which requires a longer length that is detrimental to the stiffness and thus stability of the system. This is especially the case when the sensor needs to be integrated in a cannula for capnography, since a cannula by itself typically has little to no stiffness.

Another advantage of the present disclosure is that the measurement length of the air is increased with a factor of 2, with the same mechanical dimensions. This facilitates integration of the sensor more easily in a cannula, and in case a patient requires oxygen supply, the counter side of the cannula can be used for this purpose as will be further described herein in connection with FIGS. 4A-4E.

FIG. 2D illustrates an exemplary alternative embodiment of fiber assembly 30 with GRIN lenses 36a and 36b instead of (a)spherical lenses 33a and 33b. GRIN lenses 36a and 36b are attractive for a capnography fiber assembly because they have a small size compatible with the cannula and can be manufactured in high volumes at low cost. Furthermore, GRIN lenses 36a and 36b can be mounted against respective optical fibers 32a and 32b enabling easy alignment, reducing interference effects and preventing spurious gas detection in the region between fiber and lens.

FIG. 2E illustrates an exemplary alternative embodiment of fiber assembly 30 with a toroidal mirror 38. A disadvantage with respect to using flat minor 35 is that now also the center of the minor 35 has to be accurately placed. An advantage of this exemplary embodiment is that no lenses with critical positioning are needed. Further, when toroidal mirror 38 is applied, optical fibers 32a and 32b need to be oriented in at a specific angle. This orientation issue is addressed by using two small wedge shaped prisms 39a and 39b, respectively, such as shown in FIG. 2F, for example.

As one having ordinary skill in the art shall appreciate in view of the teachings herein, the embodiments shown above with a single reflection can be extended to configurations with multiple reflections.

Further, as one having ordinary skill in the art shall appreciate in view of the teachings herein, other types of reflectors can be used.

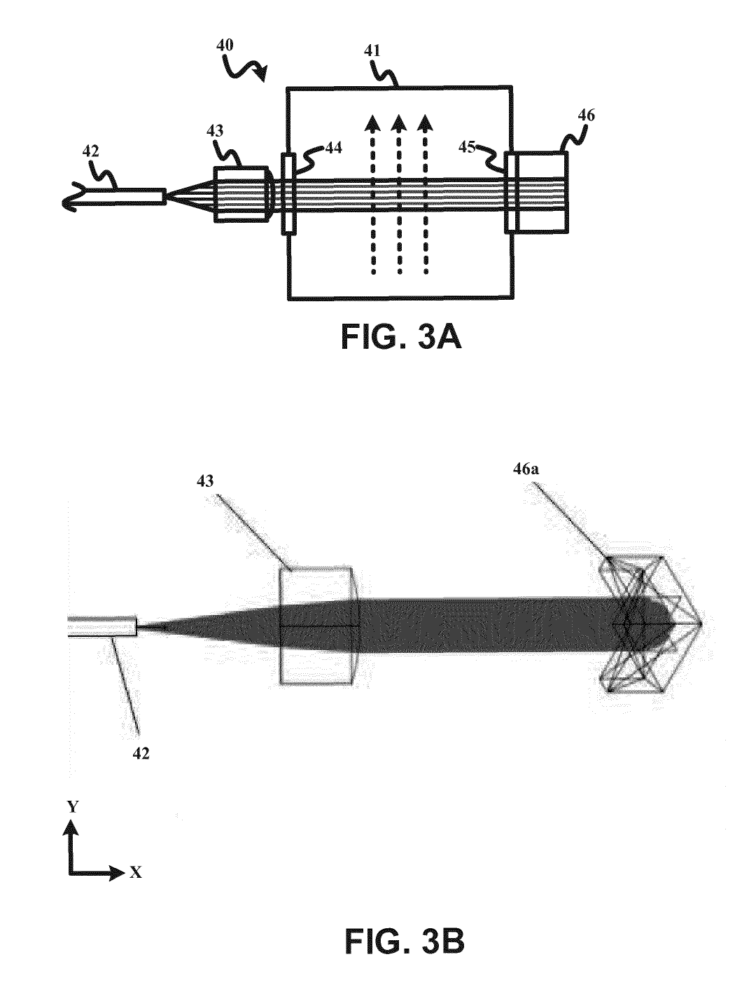

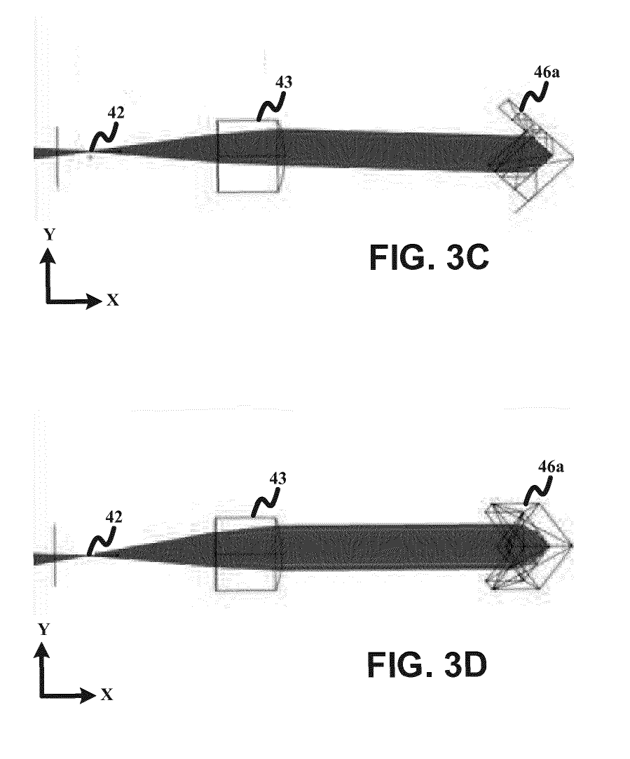

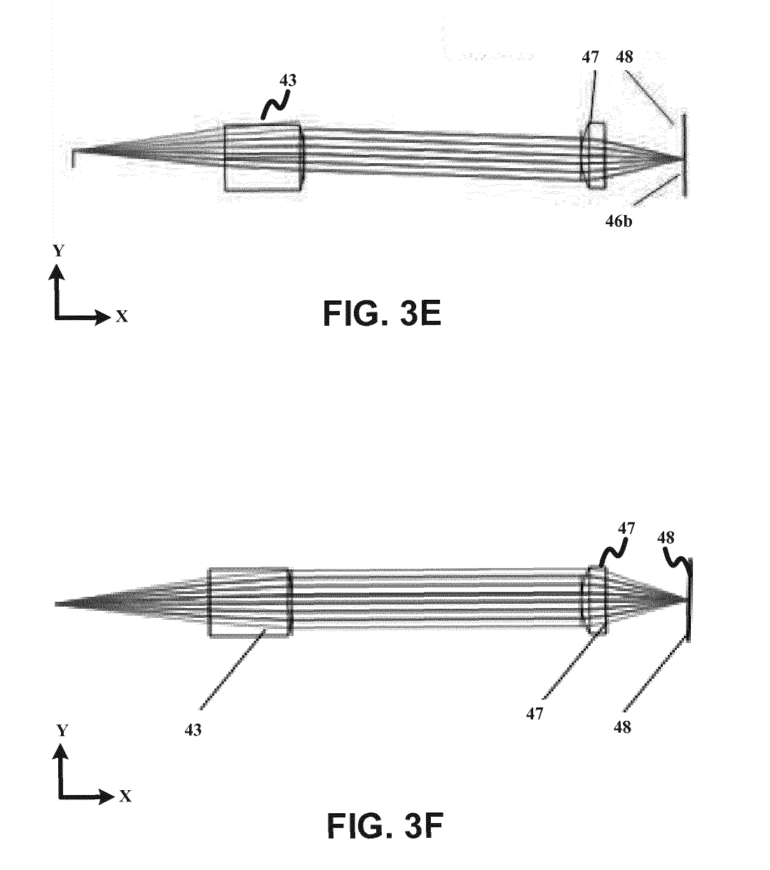

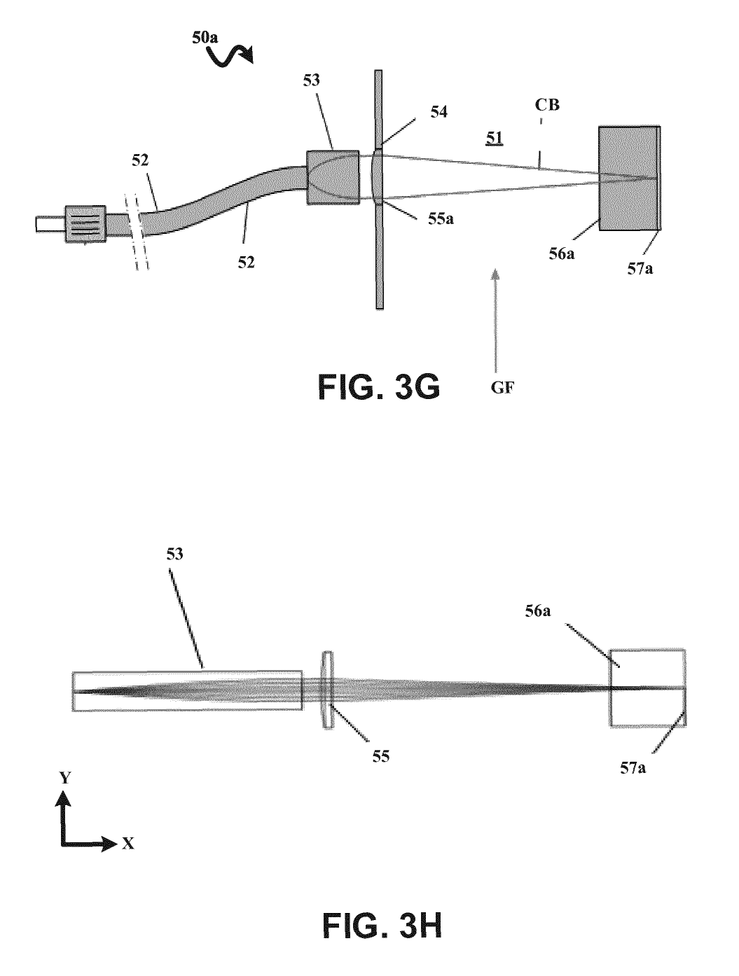

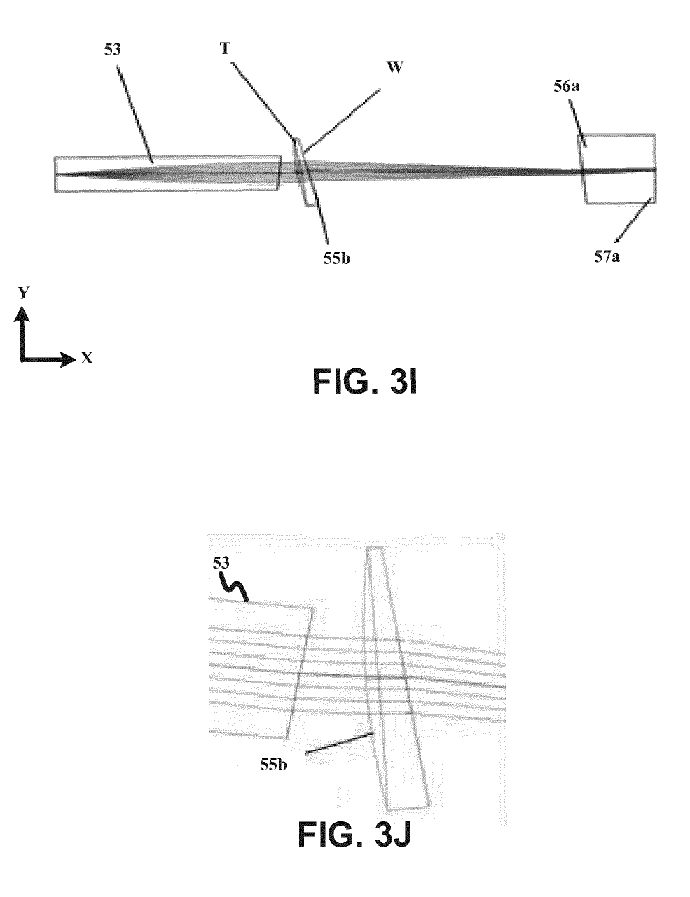

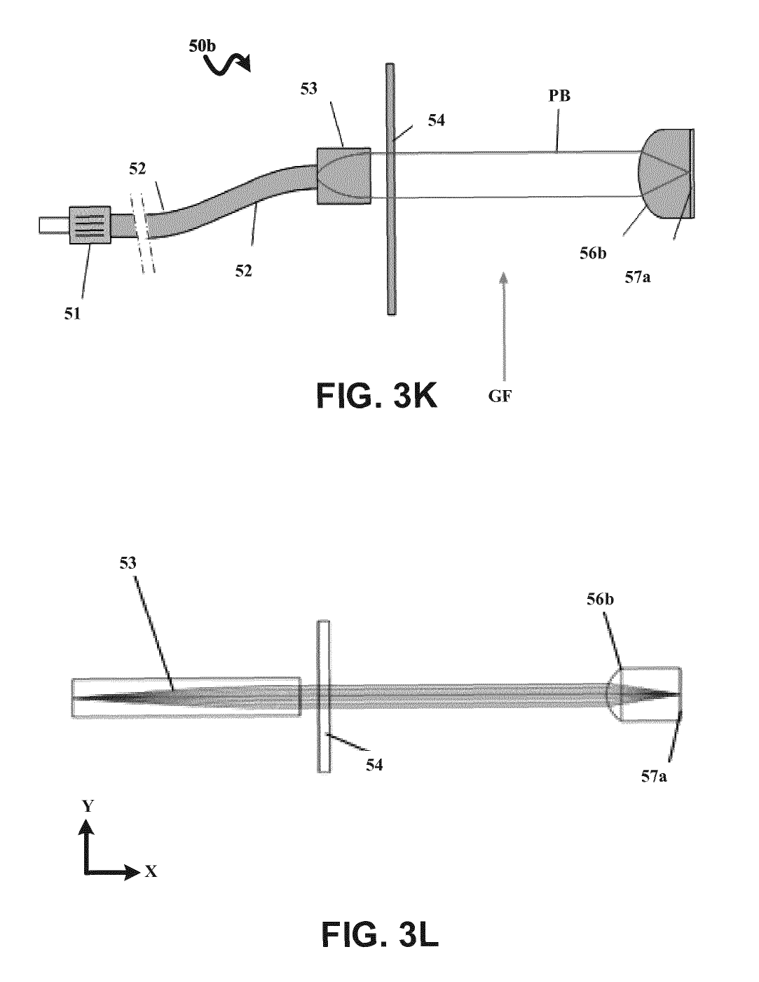

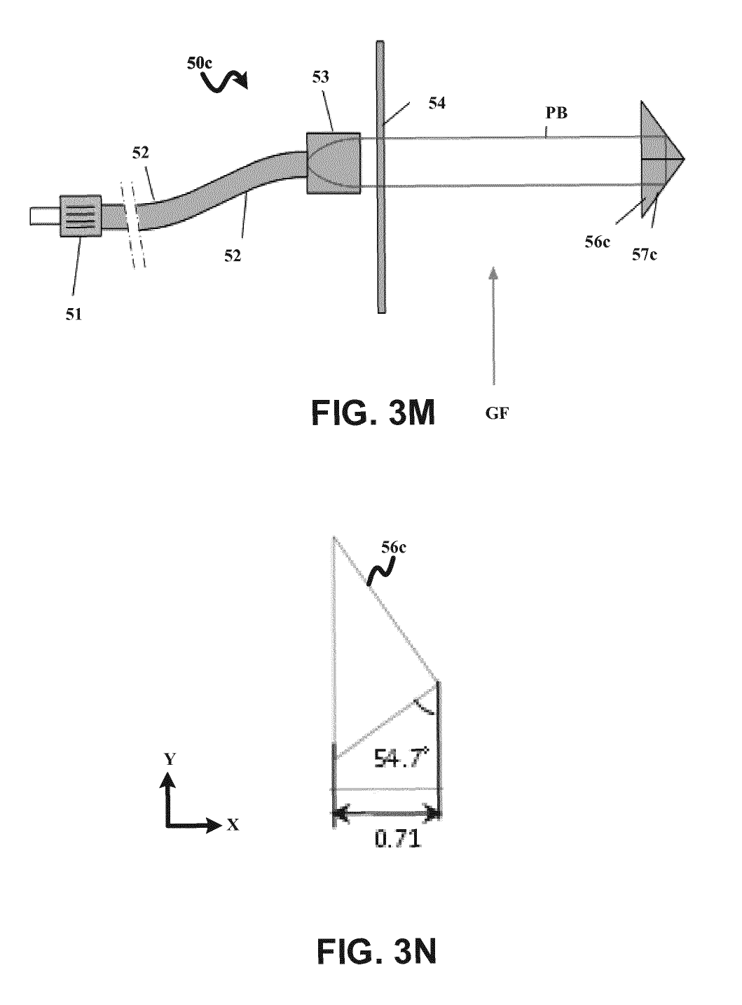

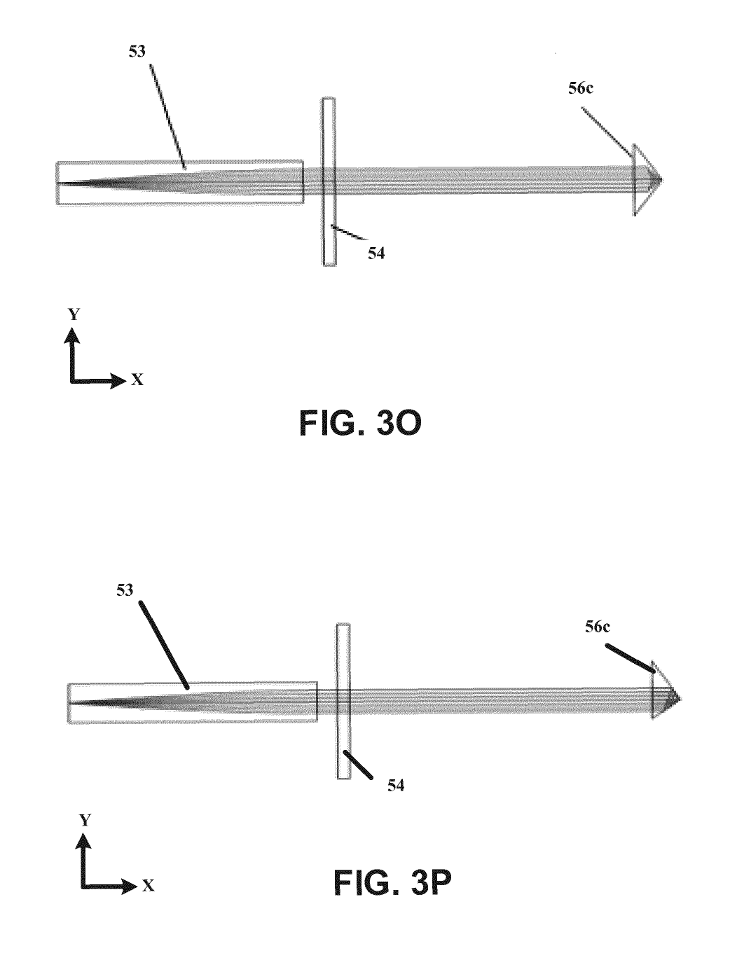

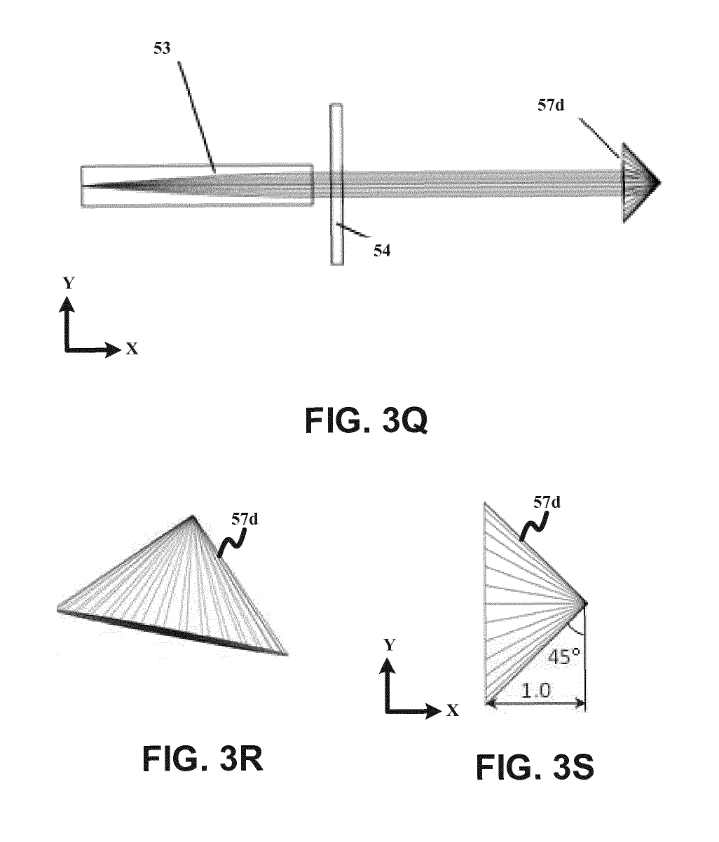

To facilitate a further understanding of the inventions of the present disclosure, the following description of FIGS. 3A-3S teaches basic inventive principles of a fiber assembly having a reflective light path between an optical fiber and a retroreflector in accordance with the inventive principles of the present disclosure. From this description of FIG. 3A-3S, those having ordinary skill in the art will appreciate how to apply the inventive principles of the present disclosure to practice numerous and various embodiments of a fiber assembly having a reflective light path between an optical fiber and a retroreflector in accordance with the inventive principles of the present disclosure.

Referring to FIG. 3A, a fiber assembly 40 employing a single mode optical fiber 42, which is a combination of a light emitting optical fiber 32a (FIG. 2A) and light receiving optical fiber 32b (FIG. 2A) in to a “two-way” fiber.

In operation, a gas containing a degree of carbon dioxide and a degree of oxygen flows through a respiratory gas detection chamber 41 as exemplary symbolized by the upwardly pointing dashed arrows. Optical fiber 42 emits a gas sensing light beam sequentially through an (a)spherical lens 43 and a window 44 across respiratory gas detection chamber 41 through a window 45 to a retroreflector 46 whereby a gas detection light beam is reflected back across respiratory gas detection chamber 41 sequentially through a window 44 and an (a)spherical lens 43 to optical fiber 42. The gas detection light beam is indicative of the degree of carbon dioxide or oxygen within any gas flowing through the respiratory gas detection chamber known in the art of the present disclosure. The wavelength of the laser source determines if the system is suitable for carbon dioxide or oxygen detection.

FIG. 3B illustrates an embodiment of fiber assembly 40 where retroreflector 46 is a rectangular corner 46a. As one having ordinary skill in the art shall appreciate in view of the teachings herein, other types and/or configurations of retroreflectors can be used in accordance with the present disclosure. However, preferably a single retroreflector is used and not an array because this leads to interferences and an increased spectral noise level.

FIGS. 3C and 3D illustrate two different examples of misalignment: FIG. 3C illustrates a lateral position error of the source of 100 μm; and FIG. 3D illustrates a lateral position error retro reflector of 100 μm. For both situations, the image of the source focus point coincides with the source focus point itself. As a result, the impact of decentering of the fiber with respect to lens, and misalignment of the retro reflector have a reduced impact on the performance of the assembly.

FIGS. 3E and 3F illustrates an exemplary embodiment of fiber assembly 40 where retroreflector 46b includes an (a)spherical lens 47 and a minor 48 (e.g., “cat's eye”). A lateral position error of the source of 100 μm (FIG. 3E) and an angular error of mirror 48 of 2° (FIG. 3F), show no shift of the image of source focus point with respect the source focus point itself.

Other embodiments with GRIN lenses or ball lenses instead of (a)spherical lens on the fiber and retroreflector side are also possible in accordance with the present disclosure as disclosed and described herein.

For example, GRIN lenses can be mounted against the fiber enabling easy alignment, reducing interference effects and preventing spurious gas detection in the region between fiber and lens.

Further, disclosed and described herein is a method for the manufacture of an exemplary fiber assembly in accordance with the present disclosure. A relatively low cost configuration becomes possible on basis of injection molded plastic components. The retroreflector can be made from an injection molded rectangular corner with a metalized surface. When a wavelength closed to 2 microns is used for CO₂ detection, single mode fibers and GRIN lenses based on silica can be applied and it is possible to use different types of plastic materials with a sufficiently low absorption of the optical parts around the gas sensing area. This plastic material can be used for a holographic lens to collimate the beam from the fiber and if necessary for a second lens in the cat's eye construction, for example. For oxygen detection at a wavelength close to 760 nm even a broader range of materials is available. Typically, a vertical cavity surface emitting laser (VCSEL) or Fabry-Perot (FP) edge emitting laser is applied as optical source.

FIG. 3G illustrates a fiber assembly 50a employing a single mode optical fiber 52, which is a combination of a light emitting optical fiber 32a (FIG. 2A) and light receiving optical fiber 32b (FIG. 2A) in to a “two-way” fiber.

In operation as a capnography device, a gas containing a degree of carbon dioxide flows through a respiratory gas detection chamber 51 as exemplary symbolized by the upwardly pointing dashed arrows. Optical fiber 52 emits a gas sensing light beam sequentially through a GRIN lens 53 and an (a)spherical lens 55a across respiratory gas detection chamber 51 to a retroreflector formed by a molded plastic 56a and a mirror 57a whereby a gas detection light beam is reflected back across respiratory gas detection chamber 51 sequentially through (a)spherical lens 55a and GRIN lens 53 to optical fiber 52. The lens 55a as positioned within a protective wall 54 combines the function of a lens and window. The gas detection light beam is indicative of the degree of carbon dioxide within any gas flowing through the respiratory gas detection chamber known in the art of the present disclosure.

More particularly for this embodiment as shown in FIG. 3H, the beam from the fiber is collimated by GRIN lens 53 (graded index lens). The GRIN lens 53 may be mounted against the fiber enabling accurate alignment, reducing interference effects and preventing spurious CO₂ gas detection in the region between optical fiber 52 and GRIN lens 53. The light beam is focused by means of the plastic (a)spherical lens 55a that is integrated in a plastic protective wall 54 to guide the air flow. The beam is focused on a metalized mirror surface 57a. The plastic volume 56a on front of the mirror 57a avoids a small focus spot at the boundary of the gas sensing volume, which may result problems due to contamination, water droplets or dirt. The plastic lens 55a has a relative long focal length. The gas flow GF is detected between the plastic lens 55a and the plastic volume 56a.

An example of the optical layout with long focal length includes a NA (numerical aperture) of the single mode fiber as 0.11. The GRIN lens having a focal length of 2.6 mm. The focal length of the plastic lens is 9 mm. The length of the cavity to measure the CO₂ in the air flow is 7.5 mm resulting in an absorption path length of 15 mm.

Further, experiments have shown that the CO₂ measurement method is very sensitive for interference effects between the optical surfaces. The interference effects can be reduced by means of an anti-reflective coating. Also, the interference effects can be avoided by tilting of the optical surfaces as depicted in FIG. 3I. Specifically, lens 55a may be replaced by a plastics lens 55b having a wedge shape. The convex lens surface of lens 55b should have a bi-conical shape or toroidal shape as best shown in FIG. 3J for a proper wavefront quality of the beam in order to couple the light back into the single mode fiber.

FIGS. 3K and 3L illustrate an alternative embodiment of a fiber assembly 50b employing a molded plastic lens 56b with minor 57a. Lens 56b has a relative short focal length, and mirror 57a is integrated with lens 56b to prevent spurious CO₂ gas detection. The gas flow is detected between the protective window 54 and the retroreflector lens 56b.

FIGS. 3M and 3N illustrate an alternative embodiment of a fiber assembly 50c employs a retroreflector prism 57c with a molded plastic 56c. The reflection for retroreflector prism 57c is based on TIR (total internal reflection). Therefore, no metalized mirror coating is necessary. FIGS. 3O and 3P show an example of the optical layout of the embodiment with molded plastic prism 56c. The NA (numerical aperture) of the single mode fiber as 0.11. The GRIN lens 53 has a focal length of 2.6 mm. The two side views at 90 degrees with respect to each other are visible in FIG. 3O and FIG. 3P.

FIGS. 3Q-3S illustrate an alternative embodiment of a fiber assembly 50c employing a retroreflector cone 57d.

In practice, embodiments of the inventions of the present disclosure with GRIN lenses, ball lenses and (a)spherical lenses on the fiber and retroreflector sides are also possible.

As described earlier herein, the fiber assemblies of the present disclosure enable a mechanical stable construction because a compactness of a housing results in a stiff system.

FIGS. 4A and 4B illustrate a compact fiber assembly 60a for capnography employing a housing 61a, a single mode optical fiber 63, a collimator 64 and a retroreflector 67. More particularly, FIG. 4A illustrates an emission of a gas sensing light beam GSLB from optical fiber 63 and FIG. 4B illustrates a reception of a gas detection light beam GDLB by optical fiber 63.

The housing 61 includes a respiratory gas detection chamber 62 suitable for incorporation with an airway adapter, an oro-nasal cannula and any other device, as known in the art of the present disclosure or hereinafter conceived for performing capnography. The collimator 64 and the retroreflector 67 are rigidly disposed within the housing 61a and optically aligned within housing 61a across the respiratory gas detection chamber 62. The single mode optical fiber 63 is optically aligned with the collimator 64 within or external to the housing 61a. The optical alignment between optical fiber 63 and collimator 64 may be achieved by an optical coupling of optical fiber 63 to collimator 64, or a mounting of optical fiber 63 onto collimator 64. In operation, the single mode optical fiber 63 emits a gas sensing light beam GSLB (FIG. 4A) through the collimator 64 across the respiratory gas detection chamber 62 with optical windows 65 and 66 to the retroreflector 67, and the single mode optical fiber 63 receives a gas detection light beam GDLB (FIG. 4B) reflected from the retroreflector 67 across the respiratory gas detection chamber 62 windows 65 and 66 through the collimator 64 to the single mode optical fiber 63. The gas detection light beam GDLB is indicative of the degree of carbon dioxidie or oxygen within any gas flowing through the respiratory gas detection chamber 62 as known in the art of the present disclosure.

FIG. 4C illustrate a compact fiber assembly 60b as a modification of fiber assembly 60a (FIGS. 4A and 4B) whereby collimator 64 is detachably attachable to a housing 61b as symbolized by the bi-directional arrow. For this embodiment, optical fiber 63 is mounted to collimator 64 to form a detachable patch cable attachable to a disposable cannula formed by housing 61b with retroreflector 67 rigidly disposed therein. Thus, in practice, the disposable cannula may be a low cost injection molded component while the more expensive patch cable may be reused for several patients and costs can be shared. Alternatively, retroreflector 67 may also be detachable attachable to housing 61b.

FIG. 4D illustrates a compact fiber assembly 70 employing an housing 71 having a respiratory gas detection chamber 72, and further employing optical fibers 73 and 74 for a folded path embodiment as shown.

An advantage of the inventions of the present disclosure is in contrast with a ‘cross over’ system such as shown in FIGS. 1A and 1B, which requires a longer length that is detrimental to the stiffness and thus stability of the system. This is especially the case when the sensor needs to be integrated in a cannula for capnography, since a cannula by itself typically has little to no stiffness.

Another advantage of the present invention is that the measurement length of the air flow in is increased with a factor of 2, with the same mechanical dimensions. This facilitates integration of the sensor more easily in a cannula, and in case a combination of capnography and oxygen supply is needed, one side of the cannula can be used for capnography and the other side for oxygen supply, as illustrated in FIG. 4E, for example.

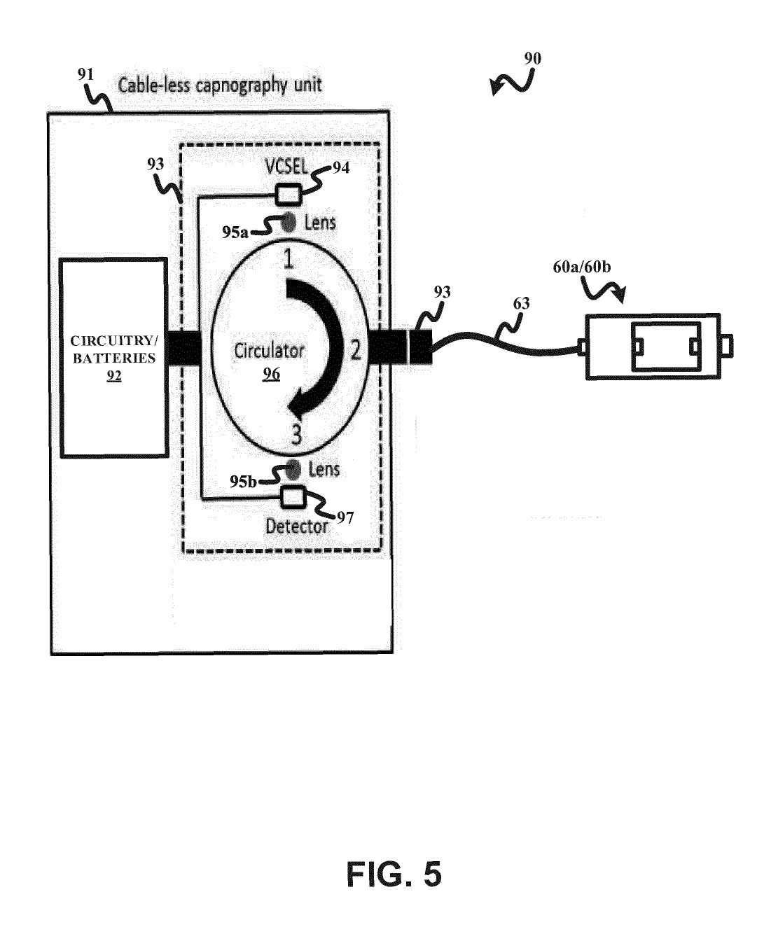

FIG. 5 illustrates a capnography device 90 employing a fiber circulator 96 for directing a carbon dioxide sampling light from the VCSEL laser 94 via a lens 95a at a port 1 to two-way optical fiber 63 at a port 2 and directs the reflected gas detection light beam from fiber assembly 60a or 60b towards a detector 97 via a lens 95b at a port 3. The circulator 96 prevents a reflection of any light back into VCSEL 94, enabling a stable single mode behavior of the VCSEL 94 during operation. To reduce spectral noise due to interference effects of the forward and backward beams, a quarter lambda plate can be incorporated at port 2 of the circulator 96, at the end of optical fiber 63 of just before the retroreflector. Also shown is circuitry/batteries 92 for driving the VCSEL, signal processing and for a wireless connection to a remote monitoring device.

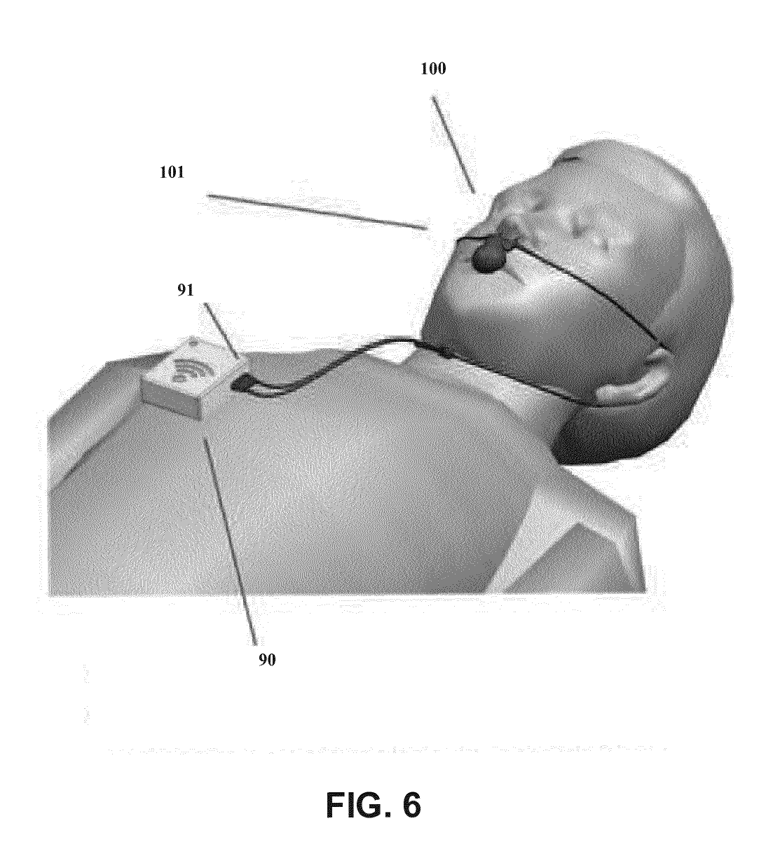

As shown in FIG. 6, respiratory gas detection device 90 provides for a reusable cableless unit with a fiber connection 91 with a fiber assembly 100 being disposed within an oro-nasal sampling cannula 101.

By way of non-limiting illustrative example, in some embodiments it is contemplated for the fiber assembly described herein to be a component of the Respironics® LoFlo® Sidestream CO₂ sensor in place of a sampling bench which uses a non-dispersive infrared (NDIR) single beam optical measurement technique to measure CO₂ and which includes a pump for drawing respiratory gas into a sample cell.

It will be further appreciated that the disclosed fiber assembly embodiments may be employed in conjunction with other types of respiratory gas sensors that are designed to sense other respired gas components such as oxygen partial pressure or concentration.

The invention disclosed herein has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

Further, as one having ordinary skill in the art shall appreciate in view of the teachings provided herein, features, elements, components, etc. disclosed and described in the present disclosure/specification and/or depicted in the appended Figures may be implemented in various combinations of hardware and software, and provide functions which may be combined in a single element or multiple elements. For example, the functions of the various features, elements, components, etc. shown/illustrated/depicted in the Figures can be provided through the use of dedicated hardware as well as hardware capable of executing software in association with appropriate software. When provided by a processor, the functions can be provided by a single dedicated processor, by a single shared processor, or by a plurality of individual processors, some of which can be shared and/or multiplexed. Moreover, explicit use of the term “processor” or “controller” should not be construed to refer exclusively to hardware capable of executing software, and can implicitly include, without limitation, digital signal processor (“DSP”) hardware, memory (e.g., read only memory (“ROM”) for storing software, random access memory (“RAM”), non-volatile storage, etc.) and virtually any means and/or machine (including hardware, software, firmware, combinations thereof, etc.) which is capable of (and/or configurable) to perform and/or control a process.

Moreover, all statements herein reciting principles, aspects, and exemplary embodiments of the present disclosure, as well as specific examples thereof, are intended to encompass both structural and functional equivalents thereof. Additionally, it is intended that such equivalents include both currently known equivalents as well as equivalents developed in the future (e.g., any elements developed that can perform the same or substantially similar functionality, regardless of structure). Thus, for example, it will be appreciated by one having ordinary skill in the art in view of the teachings provided herein that any block diagrams presented herein can represent conceptual views of illustrative system components and/or circuitry embodying the principles of the invention. Similarly, one having ordinary skill in the art should appreciate in view of the teachings provided herein that any flow charts, flow diagrams and the like can represent various processes which can be substantially represented in computer readable storage media and so executed by a computer, processor or other device with processing capabilities, whether or not such computer or processor is explicitly shown.

Having described preferred and exemplary embodiments of fiber assembly for capnography, (which embodiments are intended to be illustrative and not limiting), it is noted that modifications and variations can be made by persons having ordinary skill in the art in view of the teachings provided herein, including the appended Figures and claims. It is therefore to be understood that changes can be made in/to the preferred and exemplary embodiments of the present disclosure which are within the scope of the present disclosure and exemplary embodiments disclosed and described herein.

Moreover, it is contemplated that corresponding and/or related systems incorporating and/or implementing the device or such as may be used/implemented in a device in accordance with the present disclosure are also contemplated and considered to be within the scope of the present disclosure. Further, corresponding and/or related method for manufacturing and/or using a device and/or system in accordance with the present disclosure are also contemplated and considered to be within the scope of the present disclosure.

1. A fiber assembly for respiratory gas detection, the fiber assembly comprising:
a housing including a respiratory gas detection chamber;
a collimator rigidly disposed within or detachably attached to the housing;
a retroreflector rigidly disposed within or detachably attached to the housing and optically aligned within the housing across the respiratory gas detection chamber with the collimator; and
a single mode optical fiber optically aligned with the collimator within or external to the housing to emit a gas sensing light beam through the collimator across the respiratory gas detection chamber to the retroreflector and to receive a gas detection light beam reflected from the retroreflector across the respiratory gas detection chamber through the collimator to the single mode optical fiber,
wherein the gas detection light beam is indicative of the degree of carbon dioxide or oxygen within any gas flowing through the respiratory gas detection chamber.

2. The fiber assembly of claim 1, wherein the collimator is a GRIN lens.

3. The fiber assembly of claim 1, wherein the retroreflector includes:
a mirror having a reflection surface; and
a lens optically aligned with the reflection surface.

4. The fiber assembly of claim 1, wherein the retroreflector includes:
a mirror having a reflection surface; and
a molded plastic enclosing the reflective surface.

5. The fiber assembly of claim 1, wherein the retroreflector is a retroreflector rectangular corner.

6. The fiber assembly of claim 1, wherein the retroreflector is a retroreflector prism.

7. The fiber assembly of claim 1, wherein the retroreflector is a retroreflector cone.

8. The fiber assembly of claim 1, further comprising:
a lens disposed within the optical alignment of the collimator and the retroreflector,
wherein a transmission of the gas sensing light beam by the single mode optical fiber is sequentially through the collimator and the lens across the respiratory gas detection chamber to the retroreflector,
wherein a reception of the gas detection light beam by the single mode optical fiber is reflected from the retroreflector across the respiratory gas detection chamber sequentially through the lens and the collimator to the single mode optical fiber.

9. The fiber assembly of claim 1, wherein the lens is a spherical lens.

10. The fiber assembly of claim 1, wherein the lens is a bi-conical or toroidal titled lens.

11. A respiratory gas detection device, comprising:
a fiber assembly including
a housing including a respiratory gas detection chamber,
a collimator rigidly disposed within or detachably attached to the housing,
a retroreflector rigidly disposed within or detachably attached to the housing and optically aligned within the housing across the respiratory gas detection chamber with the collimator, and
a single mode optical fiber optically aligned with the collimator within or external to the housing to emit a gas sensing light beam through the collimator across the respiratory gas detection chamber to the retroreflector and to receive a gas detection light beam reflected from the retroreflector across the respiratory gas detection chamber through the collimator to the single mode optical fiber,
wherein the gas detection light beam is indicative of the degree of carbon dioxide or oxygen within any gas flowing through the respiratory gas detection chamber; and
an optical control assembly optically coupled to the single mode optical fiber, the optical control assembly including
a laser for generating the gas sensing light beam,
a light detector for detecting the gas detection light beam, and
an optical fiber circulator or optical fiber coupler structurally configured to direct the gas sensing light beam from the laser to the single mode optical fiber and to direct the gas detection light beam from the single mode optical fiber to the light detector.

12. The respiratory gas detection device of claim 11, wherein the collimator is a GRIN lens.

13. The respiratory gas detection device of claim 11, wherein the retroreflector includes:
a mirror having a reflection surface; and
a lens optically aligned with the reflection surface.

14. The respiratory gas detection device of claim 11, wherein the retroreflector includes:
a mirror having a reflection surface; and
a molded plastic enclosing the reflective surface.

15. The respiratory gas detection device of claim 11, wherein the retroreflector is a retroreflector rectangular corner.

16. The respiratory gas detection device of claim 11, wherein the retroreflector is a retroreflector prism.

17. The respiratory gas detection device of claim 11, wherein the retroreflector is a retroreflector cone.

18. The respiratory gas detection device of claim 11, further comprising:
a lens disposed within the optical alignment of the collimator and the retroreflector,
wherein a transmission of the gas sensing light beam by the single mode optical fiber is sequentially through the collimator and the lens across the respiratory gas detection chamber to the retroreflector,
wherein a reception of the gas detection light beam by the single mode optical fiber is reflected from the retroreflector across the respiratory gas detection chamber sequentially through the lens and the collimator to the single mode optical fiber.

19. The respiratory gas detection device of claim 11, wherein the lens is a spherical lens.

20. The respiratory gas detection device of claim 11, wherein the lens is a bi-conical toroidal titled lens.
